(19) United States
(12) Patent Application Publication
(10) Pub. No.: US 2015/0165195 A1
(43) Pub. Date: Jun. 18, 2015

(54) DEVICE FOR THE APPLICATION OF A TRANSCUTANEOUS ELECTRICAL STIMULATION STIMULUS

(71) Applicant: cerbomed GmbH, Erlangen (DE)

(72) Inventors: Andreas HARTLEP, Holzkirchen (DE); Wolf Gerhard FRENKEL, Inzigkofen-Engelswies (DE); Thomas ZSCHAECK, Nuernberg (DE); Martin HYCA, Erlangen (DE)

(21) Appl. No.: 14/567,523

(22) Filed: Dec. 11, 2014

(30) Foreign Application Priority Data
Dec. 17, 2013 (DE) 10 2013 021 175.1

(51) Int. Cl. A61N 1/08; A61N 1/36

(52) U.S. Cl. CPC A61N 1/08; A61N 1/36014; A61N 2001/083

(57) ABSTRACT

A device for the application of a transcutaneous electric stimulation stimulus onto the surface of a section of the human ear, which includes a number of electrodes, which are arranged at or in an electrode carrier. The device includes a controlling device which controls or closed-loop controls the creation of a potential difference between the electrodes. The device has a signal device which displays or indicates the resistance which exists between the electrodes at generated potential difference when the device is arranged at or in the ear. The signal device includes at least one signal element which is switched in dependence of the measured resistance. To be able to adjust the stimulation quality of the transcutaneous stimulation by the user the device in an optimal manner the signal device has a plurality of signal elements. Different signal elements are switched in dependence on pre-determined bandwidths of the resistance.

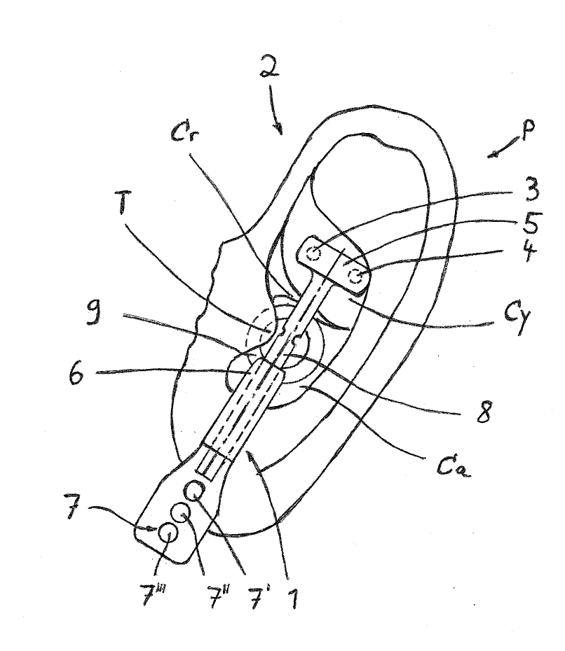

The present application claims priority of DE 10 2013 021 175.1, filed Dec. 17, 2013, the priority of this application is hereby claimed and this application is incorporated herein by reference.

### BACKGROUND OF THE INVENTION

The invention relates to a device for the application of a transcutaneous electric stimulation stimulus onto the surface of a section of the human ear, which comprises a number of electrodes, which are arranged at or in an electrode carrier, wherein the device comprises a controlling device which controls or closed-loop controls the creation of a potential difference between the electrodes, wherein the device comprises signal means which display or indicate the resistance which exists between the electrodes at generated potential difference when the device is arranged at or in the ear, wherein the signal means comprise at least one signal element which is switched in dependence of the measured resistance.

It is generally known to take influence on the neurophysiological and neuroelectrical quality through invasive and non-invasive stimulation of the nerves and thereby on the function of the simulated nerves. Hereby different conditions of sickness can be treated. Numerous devices exist both for the invasive and the non-invasive stimulation.

The present invention is basing upon the method of the transcutaneous electrical stimulation of the nerves. At this method pulse currents of different current forms, amplitudes, pulse durations and frequencies are administered through the skin on different nerves and change their status parameter in an advantageous way.

A device of the kind mentioned above is disclosed in U.S. Pat. No. 5,514,175 A and US 2009/0082831 A1. A similar device is known from DE 10 2010 054 165 B3. Here, a device for the transcutaneous stimulation of the vagus nerve of the human body is described which electrode head with two electrodes is arranged in the region of the Cymba conchae; such a positioning of the electrodes has been proven as being beneficial. Thereby, the region of the Cymba conchae is that region of the concha of the ear which is located above the Crus helicis; it is also called Hemiconcha superior. Then, below the Crus helicis downwards the region of the Cavum conchae extends. Finally, also DE 10 2011 018 228 A1 shows a similar solution.

It has been proven as being difficult to arrange the electrode head with the electrodes in the ear in such a manner that an optimized contact quality is given for the transcutaneous stimulation. A contact of the electrodes is not only optimized which causes no too high resistance between the electrodes; also, a too low resistance between the electrodes is detrimental and thus not optimal.

### SUMMARY OF THE INVENTION

Thus, it is an object of the present invention, to supply a device of the mentioned kind by which it is possible for the physician or the patient by himself in a simple manner to position the stimulation device in the ear in such a way that an optimal contact quality for the stimulation can be obtained.

The solution of this object by the invention is characterized in that the signal means comprise a plurality of signal elements, wherein different signal elements are switched in dependence on pre-determined bandwidths of the resistance.

Thereby, the signal means can be firmly connected with the device; but it is also possible that the signal means are separable or are separated from the device and that the controlling device and the signal means can communicate via a wire or via a wireless connection with another. In the latter case the signal means can be separated from the device and can be better observed during the search of an optimal position for the transcutaneous stimulation.

Furthermore, it is beneficial for a good observation of the signal means during the arrangement of the electrodes on the skin surface when according to a further development the electrode carrier with the electrodes at the one hand (thus the otoplastic) and the controlling device at the other hand (thus the stimulator as such) are designed as separate parts which are in connection with another, wherein the signal means are firmly connected with the controlling device.

Thereby, the signal elements can be light emitting diodes, wherein the light emitting diodes emit preferably light of different colour. Thereby, three light emitting diodes can be arranged. The three light emitting diodes can emit green, yellow and red light and serve as “traffic light” (see below). Also an equipment with five different coloured light emitting diodes has been proven so that it is possible to recognize the direction of the optimization in an easier manner.

The signal means can also comprise a signal element for displaying of a cipher. Hereby, a cipher between “0” and “9” can be displayed to be able to evaluate the contact quality.

The at least one signal element can also be, according to another embodiment, an acoustic sender; hereby, especially a piezoelectric crystal is considered. The control device can thereby be designed to control the acoustic sender in dependence on the resistance which exists between the electrodes to emit different frequencies.

The at least one signal element can also be, according to another embodiment, a sender which emits a signal which can be sensed by the user of the device. Hereby, especially a sender is considered which emits vibrations. Accordingly, tactile stimuli could then be recognized by the user of the device which deliver information concerning the contact quality (such solutions are known as vibration means as used in mobile phones).

As a further alternative embodiment it is possible that said signal element is designed as screen on which information is given as plaintext concerning the change of the position of the electrodes on the skin.

According to the invention generally the optimization of the stimulation quality is concerned which should be achieved in such manner that a feedback concerning the contact quality is given to the user respectively patient of the device from the device. Thereby, according to a preferred embodiment of the invention also hints are given to the user how he or she if applicable should alter the position of the electrodes on the surface of the skin to obtain a better result.

The proposed device can thereby work due to a preferred embodiment like a “traffic light”, wherein in this case e.g. three light emitting diodes are arranged which signal the status of the contact quality. Thereby, three different colours can be provided (“green”-“yellow”-“red”), wherein for sake of the display of an intermediate status also two light emitting diodes can glow simultaneously (namely “green and yellow” or “yellow and red”). This delivers a hint to the user if a re-positioning of the electrodes on the skin surface leads to an improved or to a deteriorated contact quality.

When the “traffic light” at first shows “red” the user displaces the electrode head on the skin surface—quite also by means of “trial and error”—until “yellow” is (also) glowing. The user can then further extrapolate the done change of position to see if after the (complete) extinguishing of “red” then “green” is (also) glowing. If “green” is glowing the user can be sure that an optimal basis is given for the subsequent stimulation.

Generally, it is also possible that not only several lamps (LEDs) with different colours are used as signal means; also, only one light emitting diode can be used which can glow in different colours. Furthermore, it can be provided that the intensity of the glowing is varied to deliver a respective signal.

The “traffic light” can also be realized in such a way that it is shown on a (colour) display. Alternatively, also a bar instead of a “traffic light” can be displayed—in this case for example by using a monochrome display—which is longer or shorter in dependence on the measured resistance.

With respect to the finding of an improved position of the electrodes on the skin surface inter alia the following measures can be taken from the user:

The change of the position of the pair of electrodes relatively to the skin—especially in the region of the Cymba conchae—can comprise a translational movement of the electrode head; this movement can be provided generally in all directions on the skin surface and parallel to the same. Then, the location of the stimulation can be changed at one location by circular swivelling of the electrodes respectively electrode carrier. Also, a pivoting of the plane, which is formed by the electrodes, can be done relatively to the plane of the skin to obtain a higher degree of parallelism to the skin surface. Also, a change of the contact pressure of the electrodes on the skin—mostly by an increase—can be considered.

This can also be done in connection with other flanked measures: So, a degreasing of the skin surface can be carried out. On the other hand also the application of a contact medium in an appropriate manner can be considered (in this case a sufficient amount of contact medium has to be chosen to obtain an improvement of the contact; at the other hand also too much contact medium must be avoided to prevent a “short circuit” between the electrodes via the skin surface which would erode the aimed therapeutic effect).

Also, it can be provided that the electrodes are equipped with structural means for the increase respectively for the homogenization of the contact conditions (e.g. a “cover” for the electrode carrier).

Thereby, the display by means of the signal means respectively signal elements according to the invention deliver as a result an indicator with respect to the electrical resistance between the electrodes of the stimulation device (respectively for its reciprocal, i.e. for the electrical conductivity).

Thereby, also a display of the voltage can occur—which results again via the Ohm's law to the magnitude of the resistance between the electrodes—which voltage must be applied for the creation of a defined current (i.e. of a testing current) between the electrodes.

So, for example a testing current of 100 μA can be taken as a basis and the voltage can be displayed by the signal elements which is necessary for reaching said testing current. Then, via the Ohm's law (U=R×I) a statement can be made concerning the present resistance respectively the impedance.

If a too high voltage is necessary (above a predetermined limit) the signal means deliver the information “no contact”-“red traffic light” (i.e. too high resistance). However, the device would also show the “red traffic light” if the current and thus the resistance is too low, i.e. if a “short circuit” is given.

For the “traffic light” can then for example at the mentioned testing current of 100 μA a range can be defined:
green: till 40 V
yellow: 40 to 60 V
red: above 60 V

As mentioned the optimal stimulation range must be limited not only upwards (in the direction of high voltages) but also downwards for which reference is made to the above explanations with respect to the “short circuit”. If namely the contact quality is too good, possibly already prior to the contact with the ear (for example if too much contact gel was applied which establishes an electrical conductive connection between the electrodes even prior the insertion in the ear), the user of the device should get also hints for the optimization, analogue to too high voltage, then however also at too low voltage.

Thus, that means the following: The establishment of beneficial conditions for an efficient transcutaneous stimulation can be defined to one side clearly. For this a defined voltage can be declared which must be applied for reaching a testing current. In this case the resistance lies until an allowed limit in the ranges which is therapeutical effective, which can be read from the required voltage for reaching the set current.

In the case of disadvantageous conditions for an effective transcutaneous stimulation two cases must be distinguished.

On the one hand the resistance (corresponding to the required voltage for reaching of a set current) can be too high because the contact quality between the skin and the electrode is not sufficient.

On the other hand the resistance can also be too low; then, a short circuit exists via the skin surface from the stimulation to the reference electrode without that the skin and so the receptive areas of the nerves are flown through the current.

Accordingly, as a further development of the proposed concept it can be provided that the “traffic light” does not only comprise three colours (“green”-“yellow”-“red”) by which generally only a too bad and a too good conductivity can be shown. Additional, it can be provided that two further light emitting diodes are used which differentiate the traffic light constellation “red” concerning “too high resistance” and “too low resistance” in the above meaning. In this case the “traffic light” would be provided with five lights.

In this connection the following should be mentioned: The measured resistance values can comprise variances in dependence on the specific circumstances and on the individual user, so that it is sometimes difficult to determine previously, i.e. prior a specific application, value ranges for the resistance respectively contact voltage which are shown by means of display elements. Accordingly, a further embodiment provides that the display ranges for the measures values (i.e. in the case of the “traffic light”: green, yellow or red) are determined individually respectively at least adapted, i.e. adjusted. The system would be in so far adaptive respectively it can adjust itself to specific circumstances in the meaning of a self-learning software. Hereby, it can thus be provided that for the operation of the stimulation device a software is employed which is “self-learning” and carries out respective adjustments automatically.

Hereby, it can be of course helpful instead of a three-element display (as in the case of the classical “traffic light”) to realize a finer subdivision—as the case may be a significant finer one. This can be developed till that status that a value between “0%” and “100%” is displayed which allows more precise conclusions on the contact quality.

Because the display changes in real-time the user of the device respectively the physician would obtain permanently a hint if he or she is on the right way in the search for an optimal positioning of the electrodes.

### BRIEF DESCRIPTION OF THE DRAWING

In the drawing an embodiment of the invention is depicted. The only FIGURE shows the view of a pinna (ear conch) in which a device for the application of a transcutaneous stimulation stimulus is inserted.

### DETAILED DESCRIPTION OF THE INVENTION

In the FIGURE a device 1 for the transcutaneous stimulation of a section of the human ear 2 is depicted. The device 1 comprises a holding rod 8 which is longitudinally movable in a base part which also comprises a controlling device 6. At the base part a resting part 9 is arranged. The general positioning of the device 1 in the ear 2 results from the indication of the substantial parts of the ear 2, namely the Pinna P with the Cavum conchae Ca, the Cymba conchae Cy, the Tragus T and the Crus helicis Cr.

At one of the ends of the holding rod 8 an electrode carrier 5 is arranged which comprises two electrodes 2 and 4 between which a potential difference is created for the purpose of a transcutaneous stimulation.

Insofar the device 1 corresponds at first to pre-known solutions, wherein especially and explicitly reference is made to DE 10 2010 054 165 B3 of the patent applicant where such a device is explained in detail.

Accordingly, the device 1 is designed to be attached in the region of the vagus nerve at the ear 2 of the person who uses the device. Thereby, a transcutaneous stimulation of the vagus nerve can be carried out.

It is essential that the device 1 comprises presently signal means 7 which display respectively indicate the electrical resistance between the electrodes 3 and 4 when the device is arranged at respectively in the ear 2. This signal means 7 comprise in the embodiment three signal elements 7′, 7″ and 7′″ which are controlled in dependence on the actual measured resistance.

The three signal elements 7′, 7″, 7′″ are three light emitting diodes of different colours in the embodiment, namely green (7′), yellow (7″) and red (7′″).

During movement of the electrode carrier 5 with the electrodes 3 and 4 the measured electrical resistance is changing so that in dependence on this—in the meaning of the above explanations—one (respectively also simultaneously two) of the light emitting diodes are controlled. So, the user can see immediately if good or at least useful conditions are given for the execution of a transcutaneous stimulation.

At “green” the contact conditions are good, the stimulation can be carried out efficiently.

At “yellow” the contact conditions deteriorate, at “red” no useful contact conditions are given.

As explained above for example two further light emitting diodes (not depicted) can be provided to indicate that in the case of non-useful contact conditions (thus at “red”) a too high or a too low resistance is given.

At a too high resistance relief can be obtained by means of contact gel, at too low resistance as the case may be a cleaning/de-greasing of the electrodes respectively of the electrode carrier (from contact gel) respectively of the skin surface must be carried out.

In this connection it can also be considered that the device sticks to the mentioned three light emitting diodes 7′, 7″ and 7′″ and the additional information concerning a too high respective a too low resistance is issued by an acoustic signal. Accordingly, it can also be provided due to the proposed concept that optical and acoustical signal elements are combined. So, by movement of the electrode carrier 5 with the electrodes 3 and 4 across the skin surface of the Cymba conchae Cy an optimal position for the stimulation can be found by regarding the signal means 7.

### LIST OF REFERENCES

1 Device for the transcutaneous stimulation
2 Ear
3 Electrode
4 Electrode
5 Electrode carrier
6 Controlling device
7 Signal means
7′ Signal element
7″ Signal element
7′″ Signal element
8 Holding rod
9 Resting part
Ca Cavum conchae
Cy Cymba conchae
T Tragus
Cr Crus helicis
P Pinna

1. A device for the application of a transcutaneous electric stimulation stimulus onto the surface of a section of the human ear, which comprises a number of electrodes, which are arranged at or in an electrode carrier, wherein the device comprises a controlling device which controls or closed-loop controls the creation of a potential difference between the electrodes, wherein the device comprises signal means which display or indicate the resistance which exists between the electrodes at generated potential difference when the device is arranged at or in the ear (2), wherein the signal means comprise at least one signal element which is switched in dependence of the measured resistance, wherein
the signal means comprise a plurality of signal elements wherein different signal elements are switched in dependence on pre-determined bandwidths of the resistance.

2. The device according to claim 1, wherein the signal means are firmly connected with the device.

3. The device according to claim 1, wherein the signal means are separable or are separated from the device and that the controlling device and the signal means can communicate via a wire or via a wireless connection with another.

4. The device according to claim 1, wherein the electrode carrier with the electrodes at the one hand and the controlling device at the other hand are designed as separate parts which are in connection with another, wherein the signal means are firmly connected with the controlling device.

5. The device according to claim 1, wherein the signal elements are light emitting diodes, wherein the light emitting diodes emit preferably light of different colour.

6. The device according to claim 5, wherein the three light emitting diodes are arranged.

7. The device according to claim 6, wherein the three light emitting diodes emit green, yellow and red light.
